(19) United States
(12) Patent Application Publication
(10) Pub. No.: US 2024/0136067 A1
(43) Pub. Date: Apr. 25, 2024

(54) METHOD, SYSTEM, AND NON-TRANSITORY COMPUTER-READABLE RECORDING MEDIUM FOR MONITORING OBJECT

(71) Applicant: Bodit Inc., Gunpo (KR)

(72) Inventors: Kwang Jae Choo, Gunpo (KR); Min Yong Shin, Suwon (KR); Heung Jong Yoo, Seoul (KR); Yoon Chul Choi, Anyang (KR); Seongjin Kim, Icheon (KR); Nayeon Kim, Icheon (KR)

(73) Assignee: Bodit Inc., Gunpo (KR)

(21) Appl. No.: 18/548,718

(22) PCT Filed: Aug. 22, 2021
(86) PCT No.: PCT/KR2021/011184

§ 371 Date: Oct. 3, 2023

(30) Foreign Application Priority Data
Mar. 4, 2021 (KR) 10-2021-0028838

(51) Int. Cl. G16H 50/20

(52) U.S. Cl. CPC G16H 50/20

(57) ABSTRACT

A method for monitoring an object is provided. The method includes the steps of: in response to information on a behavior of a domestic animal being estimated from sensor data measured by a sensor for the domestic animal using a machine learning-based behavior recognition model, estimating health status of the domestic animal with reference to the information on the behavior of the domestic animal and a health criterion for the domestic animal; and determining first breeding information on the domestic animal to be provided to a user on the basis of the health status.

### CROSS-REFERENCE TO RELATED APPLICATION(S)

This application is a national phase of Patent Cooperation Treaty (PCT) International Application No. PCT/KR2021/011184 filed on Aug. 23, 2021, which claims priority to Korean Patent Application No. 10-2021-0028838 filed on Mar. 4, 2021. The entire contents of PCT International Application No. PCT/KR2021/011184 and Korean Patent Application No. 10-2021-0028838 are hereby incorporated by reference.

### FIELD OF THE INVENTION

The present invention relates to a method, system, and non-transitory computer-readable recording medium for monitoring an object.

### BACKGROUND

In recent years, there has been a lot of research on machine learning technology, and techniques have been introduced to efficiently monitor a domestic animal such as a calf using a machine learning-based behavior recognition model.

As an example of related conventional techniques, Korean Laid-Open Patent Publication No. 10-2020-0059445 discloses a technique comprising: a data collection step for collecting data from a sensor; a feature point generation step for analyzing the collected data to generate feature points; and a pattern analysis step for passing the feature points through a modeled classifier to detect a behavior pattern.

However, the techniques introduced so far as well as the above-described conventional technique simply provide a user with information on a behavior of a monitored domestic animal, which is insufficient for efficient monitoring of multiple domestic animals.

In particular, the number of domestic animals to be managed by one person has been increasing in recent years, which makes it nearly impossible to manage each individual in detail, and it has been difficult to quickly identify health status of the domestic animals (e.g., disease infection status or growth status) and take appropriate measures by simply receiving information on behaviors of the domestic animals.

In this connection, the inventor(s) present a technique for quickly and accurately identifying health status of a domestic animal to be monitored, and situationally and appropriately providing information necessary for breeding of the domestic animal on the basis of the health status, thereby assisting in efficient management of the domestic animal.

### SUMMARY OF THE INVENTION

One object of the present invention is to solve all the above-described problems in prior art.

Another object of the invention is to: in response to information on a behavior of a domestic animal being estimated from sensor data measured by a sensor for the domestic animal using a machine learning-based behavior recognition model, estimate health status of the domestic animal with reference to the information on the behavior of the domestic animal and a health criterion for the domestic animal; and determine first breeding information on the domestic animal to be provided to a user on the basis of the health status.

Yet another object of the invention is to quickly and accurately identify health status of a domestic animal to be monitored, and situationally and appropriately provide information necessary for breeding of the domestic animal on the basis of the health status, thereby assisting in efficient management of the domestic animal.

The representative configurations of the invention to achieve the above objects are described below.

According to one aspect of the invention, there is provided a method comprising the steps of: in response to information on a behavior of a domestic animal being estimated from sensor data measured by a sensor for the domestic animal using a machine learning-based behavior recognition model, estimating health status of the domestic animal with reference to the information on the behavior of the domestic animal and a health criterion for the domestic animal; and determining first breeding information on the domestic animal to be provided to a user on the basis of the health status.

According to another aspect of the invention, there is provided a system comprising: a health status estimation unit configured to, in response to information on a behavior of a domestic animal being estimated from sensor data measured by a sensor for the domestic animal using a machine learning-based behavior recognition model, estimate health status of the domestic animal with reference to the information on the behavior of the domestic animal and a health criterion for the domestic animal; and a breeding information management unit configured to determine first breeding information on the domestic animal to be provided to a user on the basis of the health status.

In addition, there are further provided other methods and systems to implement the invention, as well as non-transitory computer-readable recording media having stored thereon computer programs for executing the methods.

According to the invention, it is possible to: in response to information on a behavior of a domestic animal being estimated from sensor data measured by a sensor for the domestic animal using a machine learning-based behavior recognition model, estimate health status of the domestic animal with reference to the information on the behavior of the domestic animal and a health criterion for the domestic animal; and determine first breeding information on the domestic animal to be provided to a user on the basis of the health status.

According to the invention, it is possible to quickly and accurately identify health status of a domestic animal to be monitored, and situationally and appropriately provide information necessary for breeding of the domestic animal on the basis of the health status, thereby assisting in efficient management of the domestic animal.

### BRIEF DESCRIPTION OF THE DRAWINGS

FIG. 1 schematically shows the configuration of an entire system for monitoring an object according to one embodiment of the invention.

FIG. 2 specifically shows the internal configuration of an object monitoring system according to one embodiment of the invention.

FIG. 3 illustratively shows how to monitor an object according to one embodiment of the invention.

FIG. 4 illustratively shows how to monitor an object according to one embodiment of the invention.

FIG. 5 illustratively shows how to monitor an object according to one embodiment of the invention.

FIG. 6 illustratively shows how to monitor an object according to one embodiment of the invention.

FIG. 7 illustratively shows how to monitor an object according to one embodiment of the invention.

FIG. 8 illustratively shows how to monitor an object according to one embodiment of the invention.

FIG. 9 illustratively shows how to monitor an object according to one embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures, and characteristics described herein may be implemented as modified from one embodiment to another without departing from the spirit and scope of the invention. Furthermore, it shall be understood that the positions or arrangements of individual elements within each embodiment may also be modified without departing from the spirit and scope of the invention. Therefore, the following detailed description is not to be taken in a limiting sense, and the scope of the invention is to be taken as encompassing the scope of the appended claims and all equivalents thereof. In the drawings, like reference numerals refer to the same or similar elements throughout the several views.

Hereinafter, various preferred embodiments of the present invention will be described in detail with reference to the accompanying drawings to enable those skilled in the art to easily implement the invention.

Although the descriptions of embodiments of the object monitoring system herein are focused on monitoring a behavior of a calf, it should be understood that the object monitoring system according to the invention may be applied to monitoring any other domestic animal such as a horse or pig.

Further, it should be understood that the behavior herein does not necessarily refer to an action of a domestic animal with movement, but may also refer to a state in which the domestic animal maintains a particular posture for a predetermined period of time without changing its posture (or with very little movement).

Configuration of the Entire System

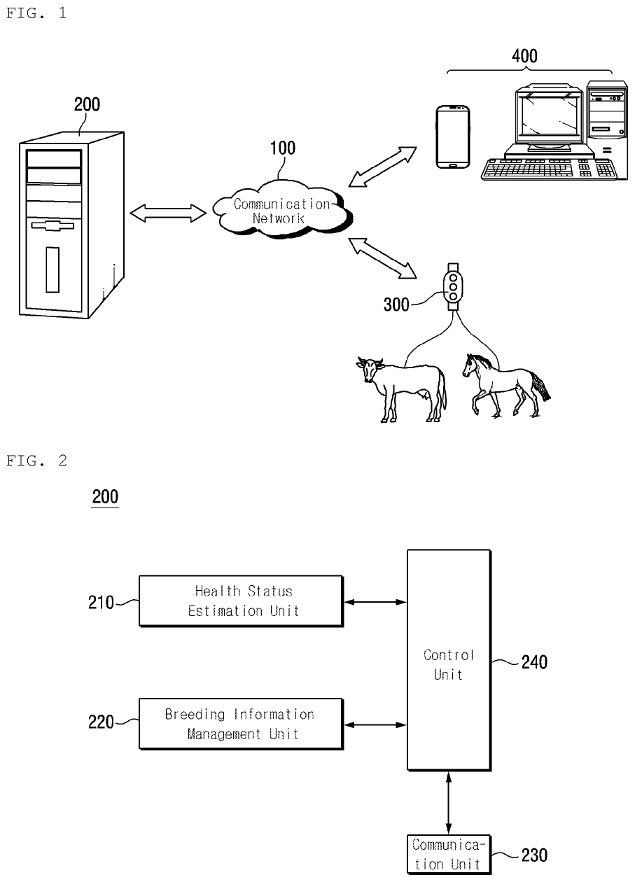

FIG. 1 schematically shows the configuration of the entire system for monitoring an object according to one embodiment of the invention.

As shown in FIG. 1, the entire system according to one embodiment of the invention may comprise a communication network 100, an object monitoring system 200, a sensor 300, and a device 400.

First, the communication network 100 according to one embodiment of the invention may be implemented regardless of communication modality such as wired and wireless communications, and may be constructed from a variety of communication networks such as local area networks (LANs), metropolitan area networks (MANs), and wide area networks (WANs). Preferably, the communication network 100 described herein may be the Internet or the World Wide Web (WWW). However, the communication network 100 is not necessarily limited thereto, and may at least partially include known wired/wireless data communication networks, known telephone networks, or known wired/wireless television communication networks.

For example, the communication network 100 may be a wireless data communication network, at least a part of which may be implemented with a conventional communication scheme such as WiFi communication, WiFi-Direct communication, Long Term Evolution (LTE) communication, 5G communication, Bluetooth communication (including Bluetooth Low Energy (BLE) communication), infrared communication, and ultrasonic communication. As another example, the communication network 100 may be an optical communication network, at least a part of which may be implemented with a conventional communication scheme such as LiFi (Light Fidelity).

Meanwhile, the communication network 100 according to one embodiment of the invention may be constructed from two or more types of communication networks. For example, the communication network between the object monitoring system 200 and the device 400 may be a high-speed wireless communication network such as LTE communication or 5G communication, or may be a wired communication network, and the communication network between the object monitoring system 200 and the sensor 300 may be a low power wide area network (LPWAN) such as LoRaWAN, SIGFOX, LTE-MTC, or narrowband Internet of Things (NB-IoT).

However, the configuration of the communication network 100 according to one embodiment of the invention is not limited to the foregoing, and may be diversely changed as long as the objects of the invention may be achieved.

Next, the object monitoring system 200 according to one embodiment of the invention may function to: in response to information on a behavior of a domestic animal being estimated from sensor data measured by a sensor for the domestic animal using a machine learning-based behavior recognition model, estimate health status of the domestic animal with reference to the information on the behavior of the domestic animal and a health criterion for the domestic animal; and determine first breeding information on the domestic animal to be provided to a user on the basis of the health status.

The configuration and functions of the object monitoring system 200 according to the invention will be discussed in more detail below.

Next, the sensor 300 according to one embodiment of the invention is digital equipment capable of connecting to and then communicating with the object monitoring system 200, and may include a known six-axis angular velocity/acceleration sensor. Thus, the sensor 300 may measure acceleration and angular velocity (i.e., the rate of tilting in a certain direction) in the X-axis, Y-axis, and Z-axis. Further, angular acceleration may be measured together with or instead of the angular velocity. The sensor 300 may be worn on or inserted in a body part (e.g., a neck) of a domestic animal (e.g., a calf). However, the type of the sensor 300 according to one embodiment of the invention and the location where the sensor 300 is worn or inserted are not particularly limited, and may be diversely changed as long as the objects of the invention may be achieved. For example, the sensor 300 according to one embodiment of the invention may include a different type of sensor other than the angular velocity and acceleration sensor, and may be inserted inside a body of a domestic animal (e.g., a calf).

In particular, the sensor 300 according to one embodiment of the invention may include an application (not shown) for assisting a user to receive services such as object monitoring from the object monitoring system 200. The application may be downloaded from the object monitoring system 200 or an external application distribution server (not shown). Meanwhile, the characteristics of the application may be generally similar to those of a health status estimation unit 210, a breeding information management unit 220, a communication unit 230, and a control unit 240 of the object monitoring system 200 to be described below. Here, at least a part of the application may be replaced with a hardware device or a firmware device that may perform a substantially equal or equivalent function, as necessary.

Next, the device 400 according to one embodiment of the invention is digital equipment capable of connecting to and then communicating with the object monitoring system 200, and any type of digital equipment having a memory means and a microprocessor for computing capabilities, such as a smart phone, a tablet, a smart watch, a smart band, smart glasses, a desktop computer, a notebook computer, a workstation, a personal digital assistant (PDAs), a web pad, and a mobile phone, may be adopted as the device 400 according to the invention.

In particular, the device 400 may include an application (not shown) for assisting a user to receive services such as object monitoring from the object monitoring system 200. The application may be downloaded from the object monitoring system 200 or an external application distribution server (not shown). Meanwhile, the characteristics of the application may be generally similar to those of the health status estimation unit 210, the breeding information management unit 220, the communication unit 230, and the control unit 240 of the object monitoring system 200 to be described below. Here, at least a part of the application may be replaced with a hardware device or a firmware device that may perform a substantially equal or equivalent function, as necessary.

Configuration of the Object Monitoring System

Hereinafter, the internal configuration of the object monitoring system 200 crucial for implementing the invention and the functions of the respective components thereof will be discussed.

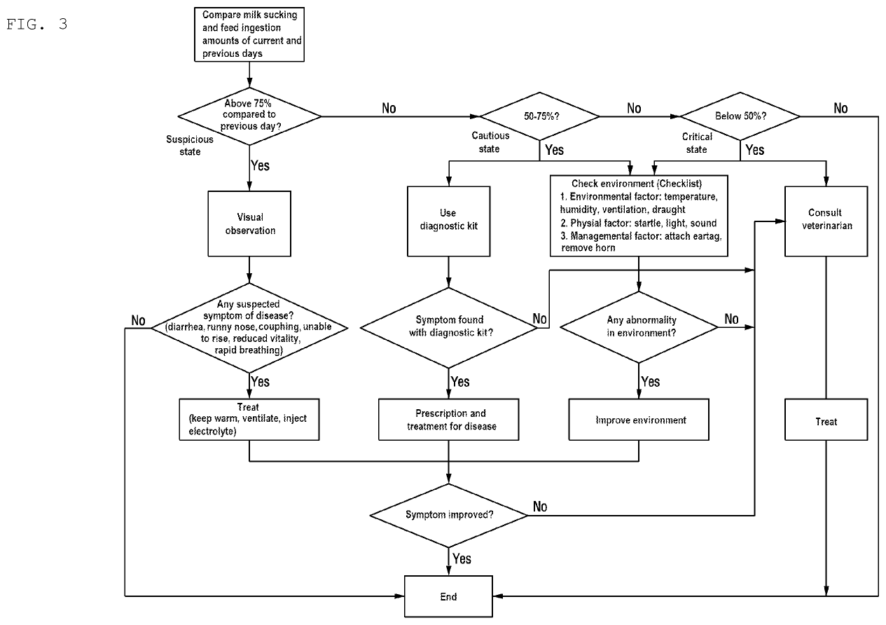

FIG. 2 specifically shows the internal configuration of the object monitoring system 200 according to one embodiment of the invention.

As shown in FIG. 2, the object monitoring system 200 according to one embodiment of the invention may comprise a health status estimation unit 210, a breeding information management unit 220, a communication unit 230, and a control unit 240. According to one embodiment of the invention, at least some of the health status estimation unit 210, the breeding information management unit 220, the communication unit 230, and the control unit 240 may be program modules to communicate with an external system (not shown). The program modules may be included in the object monitoring system 200 in the form of operating systems, application program modules, or other program modules, while they may be physically stored in a variety of commonly known storage devices. Further, the program modules may also be stored in a remote storage device that may communicate with the object monitoring system 200. Meanwhile, such program modules may include, but are not limited to, routines, subroutines, programs, objects, components, data structures, and the like for performing specific tasks or executing specific abstract data types as will be described below in accordance with the invention.

Meanwhile, the above description is illustrative although the object monitoring system 200 has been described as above, and it will be apparent to those skilled in the art that at least a part of the components or functions of the object monitoring system 200 may be implemented in the sensor 300, the device 400, or a server (not shown) or included in an external system (not shown), as necessary.

First, the health status estimation unit 210 according to one embodiment of the invention may function to, in response to information on a behavior of a domestic animal being estimated from sensor data measured by a sensor for the domestic animal using a machine learning-based behavior recognition model, estimate health status of the domestic animal with reference to the information on the behavior of the domestic animal and a health criterion for the domestic animal.

Specifically, the sensor 300 according to one embodiment of the invention may measure sensor data from at least one domestic animal. According to one embodiment of the invention, the sensor 300 may be worn on or inserted in a body part of each of the at least one domestic animal, and the sensor data may include acceleration data and/or angular velocity data. The machine learning-based behavior recognition model according to one embodiment of the invention may recognize a behavior of each domestic animal on the basis of the sensor data measured as above. Here, according to one embodiment of the invention, the behavior recognition model may be implemented using a variety of known machine learning algorithms. For example, it may be implemented using an artificial neural network such as a convolutional neural network (CNN) or a recurrent neural network (RNN), but is not limited thereto.

Further, the sensor 300 according to one embodiment of the invention may estimate information on a behavior of the domestic animal from the measured sensor data using a machine learning-based behavior recognition model. According to one embodiment of the invention, the information on the behavior of the domestic animal may include a type of a behavior performed by the domestic animal, a time of occurrence of the behavior, a duration of the behavior, a magnitude or a number of times of the behavior, and the like. Furthermore, the sensor 300 according to one embodiment of the invention may cause the health status estimation unit 210 according to one embodiment of the invention to acquire the estimated information on the behavior of the domestic animal.

For example, according to one embodiment of the invention, when the domestic animal to be monitored is a calf, the information on the behavior of the calf may include milk sucking (or an amount of milk sucking), feed ingestion (or an amount of feed ingestion), water drinking (or an amount of water drinking), rumination, an activity level, sitting, rising, failing to rise, and coughing. Further, the sensor 300 according to one embodiment of the invention may transmit the information on the behavior to the object monitoring system 200 so that the health status estimation unit 210 according to one embodiment of the invention may acquire the information on the behavior.

However, the information on the behavior of the domestic animal according to one embodiment of the invention is not limited to those listed above, and may be diversely changed as long as the objects of the invention may be achieved.

Further, the health status estimation unit 210 according to one embodiment of the invention may estimate health status of the domestic animal with reference to a health criterion for the domestic animal and the information on the behavior of the domestic animal estimated by the sensor 300 according to one embodiment of the invention.

More specifically, according to one embodiment of the invention, the health status of the domestic animal may refer to whether the domestic animal is infected with a disease, a degree of infection when the domestic animal is infected with the disease, whether the domestic animal is growing normally, a degree of growth of the domestic animal, physical activity status of the domestic animal, and the like. Further, according to one embodiment of the invention, the health status of the domestic animal may be classified into a predetermined number of states according to the health criterion for the domestic animal, and may be calculated in a format such as a score.

Here, according to one embodiment of the invention, the health criterion for the domestic animal is a predetermined criterion for determining the health status of the domestic animal, and may include, for example, an average milk sucking amount of other individuals similar to the domestic animal in terms of age (or age in days), sex, size, weight, and the like, an average feed ingestion amount of the individuals, an average activity level of the individuals, and whether a particular behavior occurs not less than a predetermined number of times. Further, according to one embodiment of the invention, the health criterion may include information on a past behavior of the domestic animal (e.g., a milk sucking amount of the domestic animal on the previous day, or an average milk sucking amount of the domestic animal over the past few days). In addition, the health status estimation unit 210 according to one embodiment of the invention may estimate the health status of the domestic animal by comparing the information on the behavior of the domestic animal with the health criterion for the domestic animal.

For example, according to one embodiment of the invention, when the domestic animal to be monitored is a calf, the health status of the calf may be classified, depending on a milk sucking amount of the calf (i.e., the information on the behavior of the calf) compared to an average milk sucking amount (i.e., the health criterion for the calf) (such an average may vary with the calf's age (or age in days), sex, size, weight, and the like), into four states such as normal (e.g., above 90% of the average), suspicious (e.g., 75-90% of the average), cautious (e.g., 50-75% of the average), and critical (e.g., below 50% of the average).

However, the information included in the health status of the domestic animal according to one embodiment of the invention or the manner of classifying the health status is not limited to the foregoing, and may be diversely changed as long as the objects of the invention may be achieved.

Meanwhile, the health status estimation unit 210 according to one embodiment of the invention may estimate the health status of the domestic animal using a machine learning-based estimation model. According to one embodiment of the invention, the machine learning-based estimation model may be a model trained using a correlation between the information on the behavior of the domestic animal and the health criterion for the domestic animal (e.g., the information on the past behavior of the domestic animal) and the health status of the domestic animal as training data.

For example, according to one embodiment of the invention, when the domestic animal to be monitored is a calf, the machine learning-based estimation model may be trained on the basis of information on a normal behavior of the calf (e.g., an activity level of the calf or a frequency or duration of a behavior of the calf such as sitting, rising, and failing to rise). Further, the health status estimation unit 210 according to one embodiment of the invention may estimate (or classify) the health status of the calf by comparing the normal behavior of the calf (i.e., the health criterion) with the information on the behavior of the calf estimated by the sensor 300 according to one embodiment of the invention, using the trained machine learning-based estimation model. Further, as will be described below, the breeding information management unit 220 according to one embodiment of the invention may determine first breeding information on the calf to be provided to a user on the basis of the estimated health status.

Meanwhile, the machine learning-based estimation model may be implemented using a variety of known machine learning algorithms. For example, it may be implemented using an artificial neural network such as a convolutional neural network (CNN) or a recurrent neural network (RNN), but is not limited thereto.

Next, the breeding information management unit 220 according to one embodiment of the invention may function to determine first breeding information on the domestic animal to be provided to a user on the basis of the health status estimated by the health status estimation unit 210 according to one embodiment of the invention.

Specifically, according to one embodiment of the invention, the first breeding information on the domestic animal may be determined for each individual according to the health status of the domestic animal. According to one embodiment of the invention, the first breeding information may include information on the behavior of the domestic animal compared to the health criterion for the domestic animal, information on actions to be taken by the user for the domestic animal, information on a diagnosis of the domestic animal provided to the user to determine second breeding information on the domestic animal to be described below, information on a breeding environment of the domestic animal, and the like.

However, the first breeding information on the domestic animal according to one embodiment of the invention is not limited to those listed above, and may be diversely changed as long as the objects of the invention may be achieved.

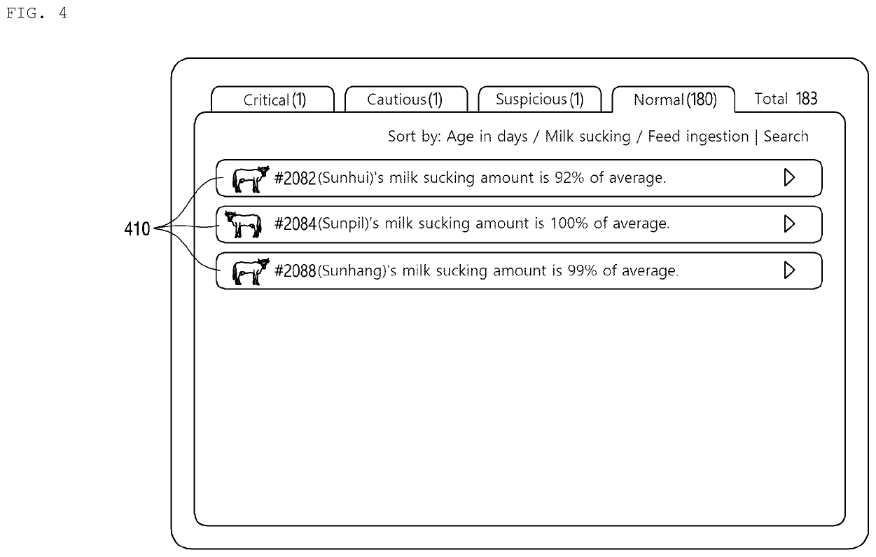

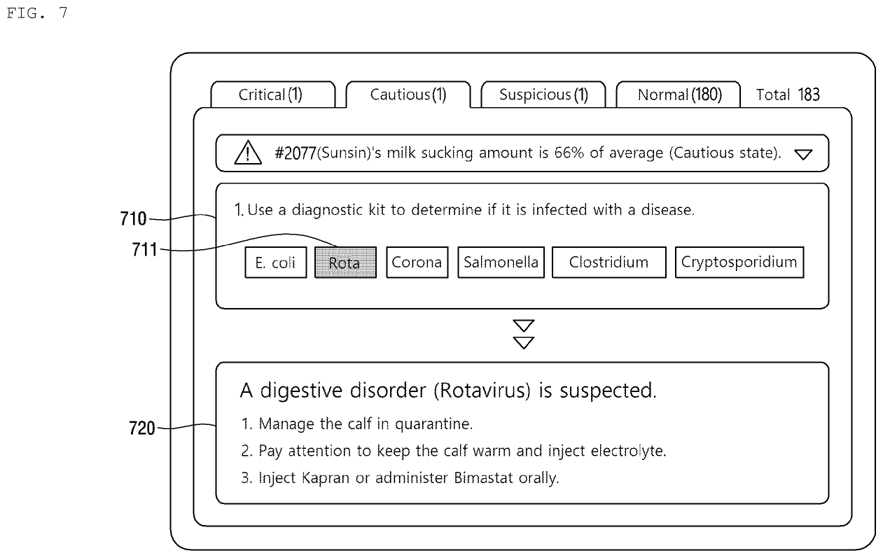

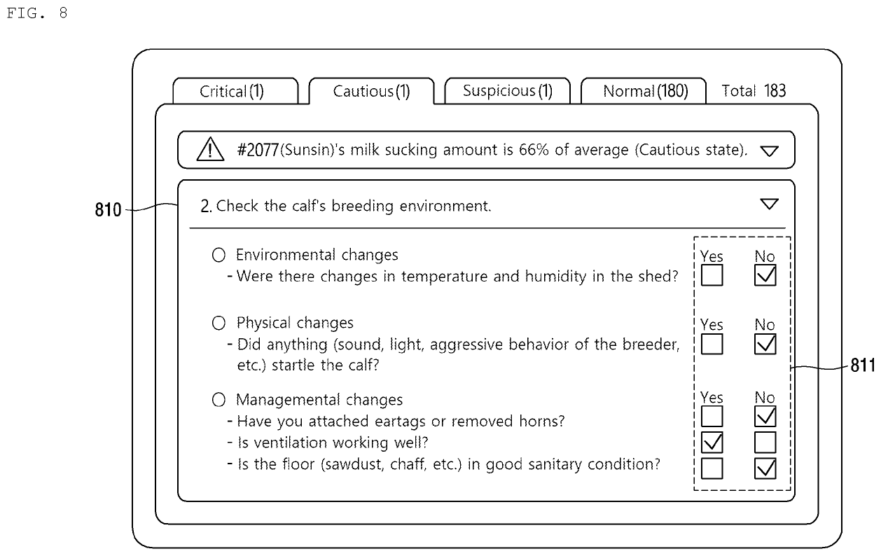

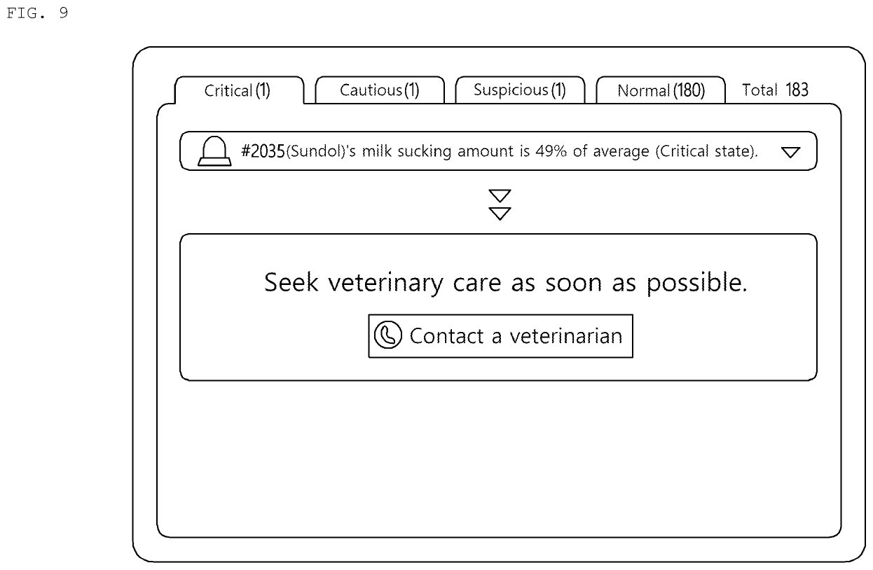

FIGS. 3 to 9 illustratively show how to monitor an object according to one embodiment of the invention.

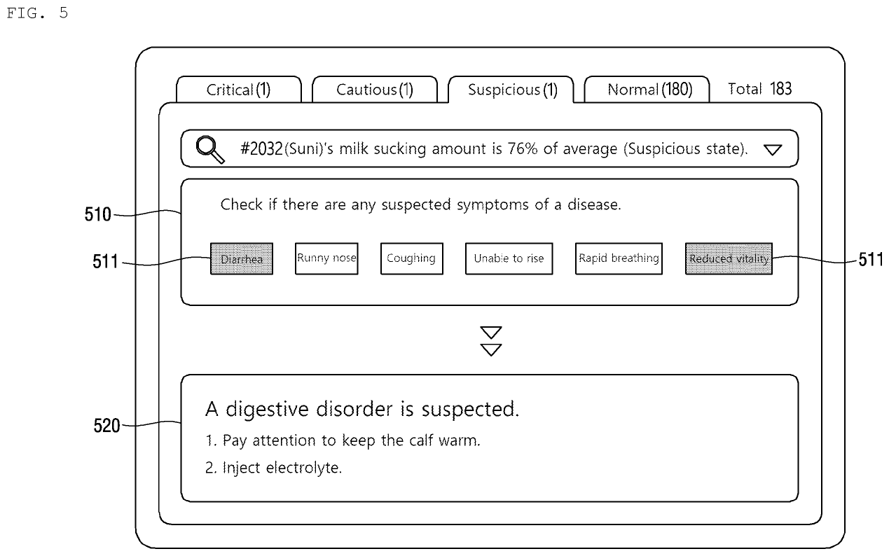

For example, referring to FIG. 4, when health status of a domestic animal corresponds to a normal state, the breeding information management unit 220 according to one embodiment of the invention may determine information on a behavior of the domestic animal compared to a health criterion for the domestic animal as first breeding information on the domestic animal 410.

As another example, referring to FIG. 9, when health status of a domestic animal corresponds to a critical state, the breeding information management unit 220 according to one embodiment of the invention may determine contact information of a veterinarian, which is information on actions to be taken by the user for the domestic animal, as first breeding information on the domestic animal. Further, when the veterinarian corresponds to the user, the breeding information management unit 220 according to one embodiment of the invention may determine contact information of a manager of the domestic animal, which is information to be provided to the veterinarian, as the first breeding information.

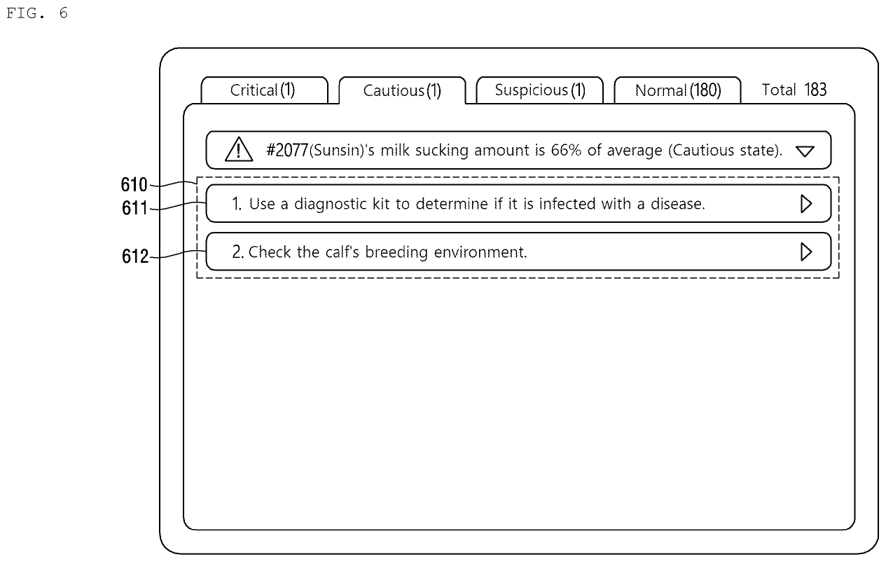

As another example, referring to FIGS. 5 to 8, when health status of a domestic animal corresponds to a suspicious or cautious state, the breeding information management unit 220 according to one embodiment of the invention may determine information on a diagnosis of the domestic animal 510, 611 and 710 and/or information on a breeding environment of the domestic animal 612 and 810 as first breeding information on the domestic animal 510, 610, 710, and 810. Further, according to one embodiment of the invention, the information on the diagnosis may include at least one suspected symptom (e.g., diarrhea, runny nose, coughing, inability to rise, rapid breathing, or reduced vitality) and/or at least one suspected causative agent (e.g., E. coli, Rota, Corona, Salmonella, Clostridium, or Cryptosporidium) of the domestic animal 510 and 710.

Here, according to one embodiment of the invention, at least one of the information on the diagnosis of the domestic animal and the information on the breeding environment of the domestic animal may be estimated by the above-described machine learning-based estimation model. That is, according to one embodiment of the invention, the machine learning-based estimation model may be a model trained using a correlation between the information on the behavior of the domestic animal and the health criterion for the domestic animal, and the information on the diagnosis of the domestic animal (and/or the information on the breeding environment of the domestic animal) as training data. Further, the breeding information management unit 220 according to one embodiment of the invention may estimate the information on the diagnosis of the domestic animal (e.g., suspected symptoms) and/or the information on the breeding environment of the domestic animal (e.g., items to be checked by the user for the domestic animal) using the machine learning-based estimation model, and determine them as first breeding information on the domestic animal, thereby assisting the user to efficiently monitor the domestic animal.

Meanwhile, the breeding information management unit 220 according to one embodiment of the invention may determine second breeding information on the domestic animal to be provided to the user, on the basis of the user's feedback on the first breeding information on the domestic animal determined as above.

Specifically, when the user inputs feedback on the first breeding information on the domestic animal, the breeding information management unit 220 according to one embodiment of the invention may determine a management method (or guideline) suitable for the health status of the domestic animal as second breeding information on the domestic animal to be provided to the user, on the basis of the feedback.

For example, referring to FIGS. 5, 7 and 8, the breeding information management unit 220 according to one embodiment of the invention may acquire feedback 511, 711 and 811 that the user inputs on the first breeding information on the domestic animal 510, 710 and 810. Then, the breeding information management unit 220 according to one embodiment of the invention may determine second breeding information on the domestic animal to be provided to the user 520 and 720 on the basis of the user's feedback 511, 711 and 811.

According to one embodiment of the invention, the above-described machine learning-based estimation model may be trained on the basis of at least one of the user's feedback on the first breeding information on the domestic animal, the second breeding information on the domestic animal, and information on a behavior of the domestic animal after the second breeding information is provided to the user. That is, according to one embodiment of the invention, the machine learning-based estimation model may be trained using a change in the health status of the domestic animal that occurs when the user manages the domestic animal according to a management method (or guidance) based on the second breeding information on the domestic animal as training data, and the health status of the domestic animal may be estimated more accurately using the trained estimation model, thereby assisting the user to efficiently monitor the domestic animal.

Next, the communication unit 230 according to one embodiment of the invention may function to enable data transmission/reception from/to the health status estimation unit 210 and the breeding information management unit 220.

Lastly, the control unit 240 according to one embodiment of the invention may function to control data flow among the health status estimation unit 210, the breeding information management unit 220, and the communication unit 230. That is, the control unit 240 according to one embodiment of the invention may control data flow into/out of the object monitoring system 200 or data flow among the respective components of the object monitoring system 200, such that the health status estimation unit 210, the breeding information management unit 220, and the communication unit 230 may carry out their particular functions, respectively.

The embodiments according to the invention as described above may be implemented in the form of program instructions that can be executed by various computer components, and may be stored on a computer-readable recording medium. The computer-readable recording medium may include program instructions, data files, and data structures, separately or in combination. The program instructions stored on the computer-readable recording medium may be specially designed and configured for the present invention, or may also be known and available to those skilled in the computer software field. Examples of the computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler, but also high-level language codes that can be executed by a computer using an interpreter. The above hardware devices may be changed to one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for monitoring an object, the method comprising the steps of:
in response to information on a behavior of a domestic animal being estimated from sensor data measured by a sensor for the domestic animal using a machine learning-based behavior recognition model, estimating health status of the domestic animal with reference to the information on the behavior of the domestic animal and a health criterion for the domestic animal; and
determining first breeding information on the domestic animal to be provided to a user on the basis of the health status.

2. The method of claim 1, wherein the first breeding information includes at least one of information on a diagnosis of the domestic animal and information on a breeding environment of the domestic animal, and
wherein at least one of the heath status, the information on the diagnosis, and the information on the breeding environment is estimated using a machine learning-based estimation model.

3. The method of claim 2, wherein the information on the diagnosis includes at least one of at least one suspected symptom and at least one suspected causative agent of the domestic animal.

4. The method of claim 1, wherein in the determining step, second breeding information on the domestic animal to be provided to the user is determined on the basis of the user's feedback on the first breeding information.

5. The method of claim 4, wherein in the estimating step, the health status is estimated using a machine learning-based estimation model, and
wherein the machine learning-based estimation model is trained on the basis of at least one of the feedback, the second breeding information, and information on a behavior of the domestic animal after the second breeding information is provided to the user.

6. The method of claim 1, wherein the health criterion includes information on a past behavior of the domestic animal, and
wherein in the estimating step, the health status is estimated by comparing the information on the behavior with the information on the past behavior.

7. A non-transitory computer-readable recording medium having stored thereon a computer program for executing the method of claim 1.

8. A system for monitoring an object, the system comprising:
a health status estimation unit configured to, in response to information on a behavior of a domestic animal being estimated from sensor data measured by a sensor for the domestic animal using a machine learning-based behavior recognition model, estimate health status of the domestic animal with reference to the information on the behavior of the domestic animal and a health criterion for the domestic animal; and
a breeding information management unit configured to determine first breeding information on the domestic animal to be provided to a user on the basis of the health status.

9. The system of claim 8, wherein the first breeding information includes at least one of information on a diagnosis of the domestic animal and information on a breeding environment of the domestic animal, and
wherein at least one of the heath status, the information on the diagnosis, and the information on the breeding environment is estimated using a machine learning-based estimation model.

10. The system of claim 9, wherein the information on the diagnosis includes at least one of at least one suspected symptom and at least one suspected causative agent of the domestic animal.

11. The system of claim 8, wherein the breeding information management unit is configured to determine second breeding information on the domestic animal to be provided to the user on the basis of the user's feedback on the first breeding information.

12. The system of claim 11, wherein the health status estimation unit is configured to estimate the health status using a machine learning-based estimation model, and
wherein the machine learning-based estimation model is trained on the basis of at least one of the feedback, the second breeding information, and information on a behavior of the domestic animal after the second breeding information is provided to the user.

13. The system of claim 8, wherein the health criterion includes information on a past behavior of the domestic animal, and
wherein the health status estimation unit is configured to estimate the health status by comparing the information on the behavior with the information on the past behavior.
